(19) United States
(12) Patent Application Publication
(10) Pub. No.: US 2022/0378676 A1
(43) Pub. Date: Dec. 1, 2022

(54) COMPOSITION FOR ENHANCING SKIN ELASTICITY OR IMPROVING SKIN WRINKLES

(71) Applicants: AMOREPACIFIC CORPORATION, Seoul (KR); KYUNGPOOK NATIONAL UNIVERSITY INDUSTRY-ACADEMIC COOPERATION FOUNDATION, Buk-gu (KR)

(72) Inventors: Hyunjung CHOI, Yongin-si (KR); Dong-Hyung CHO, Suseong-gu (KR); Ji-Eun BAE, Buk-gu (KR); Joonbum KIM, Yongin-si (KR); Ji Yeon CHOI, Changwon-si (KR); Daejin MIN, Yongin-si (KR); Hye Won NA, Yongin-si (KR); Hyoung June KIM, Yongin-si (KR); Wonseok PARK, Yongin-si (KR)

(73) Assignees: AMOREPACIFIC CORPORATION, Seoul (KR); KYUNGPOOK NATIONAL UNIVERSITY INDUSTRY-ACADEMIC COOPERATION FOUNDATION, Buk-gu (KR)

(21) Appl. No.: 17/744,529

(22) Filed: May 13, 2022

(30) Foreign Application Priority Data
May 18, 2021 (KR) 10-2021-0064167
Apr. 20, 2022 (KR) 10-2022-0048732

(51) Int. Cl. A61K 8/362; A61K 8/02; A61Q 19/08; A23L 29/00

(52) U.S. Cl. CPC A61K 8/362; A61K 8/0241; A61Q 19/08; A23L 29/035; A61K 2800/782

(57) ABSTRACT

Disclosed herein is a composition for enhancing skin elasticity or improving skin wrinkles. In one aspect, 2-isopropylmalic acid or a salt, hydrate or solvate thereof, which is an active ingredient of the present disclosure, activates skin primary cilia and inhibits the expression of MMP-1 to enhance skin elasticity and improve skin wrinkles. In addition, in one aspect, 2-isopropylmalic acid or a salt, hydrate or solvate thereof, which is an active ingredient of the present disclosure, is a safe material and has the advantage that it can be treated in the living body in various forms such as cosmetics and food.

### CROSS-REFERENCE TO RELATED APPLICATION

The present application claims priority to Korean Patent Applications No. 10-2021-0064167, filed May 18, 2021 and 10-2022-0048732, filed Apr. 20, 2022, the entire contents of which is incorporated herein for all purposes by this reference.

### TECHNICAL FIELD

The present specification relates to a composition for enhancing skin elasticity or improving skin wrinkles.

### BACKGROUND ART

The skin is the outermost organ of the human body and plays a role in protecting the human body from various external stimuli. Among the constituent layers of the skin, the epidermis plays a role in preventing the evaporation of moisture inside the human body. The epidermis is divided into stratum corneum, granular layer, stratum corneum, and basal layer in order from the outside. The cells of the stratum corneum act like bricks, and the intercellular lipids between the keratinocytes act like a mortar, forming the skin barrier.

It is known that the normal function of the skin is lowered by external stimuli such as ultraviolet rays and particulate matter and internal causes such as metabolites produced by intestinal microbes and stress hormones, which enhances the phenomenon of loss of elasticity, keratinization, and wrinkle formation. However, there are few solutions to control this.

Primary cilia are a cellular structure that plays an important role in acting as a bio-antenna for cells to adapt to changes in their surrounding environment. When these primary cilia are activated in the skin, it is known that desirable effects on the skin, such as smoothing the keratin, strengthening the skin barrier, and preventing excessive melanin production appear.

The present inventors have been searching for a method for enhancing skin elasticity and improving skin wrinkles, confirming that the primary cilia activated by 2-isopropylmalic acid is effective in enhancing elasticity and improving skin wrinkles, thereby completing the present invention.

### DISCLOSURE

### Technical Problem

In one aspect, the present disclosure is to provide a composition for enhancing skin elasticity or improving skin winkles.

### Technical Solution

In one aspect, the present disclosure provides a composition for enhancing skin elasticity or improving skin wrinkles, including 2-isopropylmalic acid or a salt, hydrate or solvate thereof as an active ingredient

In an exemplary embodiment, the composition of the 2-isopropylmalic acid or the salt, hydrate or solvate thereof may be administered at a dosage of 1.7 to 27 mg/kg/day.

In an exemplary embodiment, the composition may activate primary cilia.

In an exemplary embodiment, the composition may inhibit the expression of the MMP-1 enzyme.

In an exemplary embodiment, the skin wrinkles may be skin wrinkles caused by particulate matter.

In an exemplary embodiment, the composition may be a food composition or a cosmetic composition.

In one aspect, the present disclosure provides a composition for activating skin primary cilia including 2-isopropylmalic acid or a salt, a hydrate or a solvate thereof as an active ingredient.

In an exemplary embodiment, the activation of skin primary cilia may be activation of skin primary cilia inhibited by particulate matter.

In an exemplary embodiment, the composition may enhance the production of skin primary cilia.

In an exemplary embodiment, the composition may be a food composition, a cosmetic composition, or a pharmaceutical composition.

In another aspect, the present disclosure provides a composition for enhancing skin defense against particulate matter including 2-isopropylmalic acid or a salt, hydrate or solvate thereof as an active ingredient.

### Advantageous Effects

In one aspect, the composition according to the present disclosure has excellent effects of enhancing skin elasticity and improving skin wrinkles without side effects.

In one aspect, the composition according to the present disclosure can inhibit the expression of an MMP-1 enzyme.

In one aspect, the composition according to the present disclosure can activate skin primary cilia.

In one aspect, the composition according to the present disclosure can enhance skin defense against particulate matter.

### BRIEF DESCRIPTION OF DRAWINGS

FIG. 1 is a result of measuring the cytotoxicity of 2-isopropylmalic acid according to an embodiment of the present disclosure.

FIG. 2 is a result of observing the primary cilia model cells treated with 2-isopropylmalic acid under a fluorescence microscope according to an embodiment of the present disclosure.

FIG. 3 is a graph showing a result of measuring the production rate and length of primary cilia in the primary cilia model cells treated with 2-isopropylmalic acid according to an embodiment of the present disclosure.

FIG. 4 is a result of observing the primary cilia model cells exposed to particulate matter treated with 2-isopropylmalic acid under a fluorescence microscope according to an embodiment of the present disclosure.

FIG. 5 is a graph showing a result of measuring the production rate and length of primary cilia in the primary cilia model cells exposed to particulate matter treated with 2-isopropylmalic acid according to an embodiment of the present disclosure.

FIG. 6 is a result of measuring the c-Jun phosphorylation inhibitory effect of 2-isopropylmalic acid according to an embodiment of the present disclosure.

FIG. 7 is a result of measuring the MMP-1 expression inhibitory effect of 2-isopropylmalic acid according to an embodiment of the present disclosure.

FIG. 8 is a result of measuring the collagen production enhancing effect of 2-isopropylmalic acid according to an embodiment of the present disclosure.

FIG. 9 is a result of measuring the inflammatory cytokine inhibitory effect of 2-isopropylmalic acid according to an embodiment of the present disclosure.

FIG. 10 is a result of measuring the MMP-1 expression inhibitory effect of 2-isopropylmalic acid in an artificial skin model according to an embodiment of the present disclosure.

FIG. 11 is a graph quantifying the expression level of MMP-1 in an artificial skin model treated with 2-isopropylmalic acid according to an embodiment of the present disclosure.

### DETAILED DESCRIPTION OF THE INVENTION

Hereinafter, the present disclosure will be described in detail.

In one aspect, the present disclosure provides a composition for enhancing skin elasticity or improving skin wrinkles, including 2-isopropylmalic acid or a salt, hydrate or solvate thereof as an active ingredient.

In the present specification, 2-isopropylmalic acid may be represented by Chemical Formula 1 below.

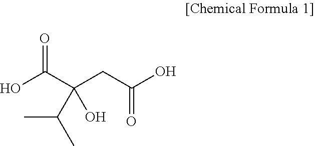

As used herein, the term “salt” means a salt according to an aspect of the present disclosure that is acceptable in medicine, cosmetics and food and has the desired activity of the parent compound. The salt may include (1) acid addition salt formed by inorganic acid such as hydrochloric acid, hydrobromic acid, sulfuric acid, nitric acid, phosphoric acid, and the like; or organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tert-butylacetic acid, lauryl sulfuric acid, gluconic acid, glutamic acid, hydroxynaphthoic acid, salicylic acid, stearic acid, and muconic acid; or (2) salt formed when an acidic proton present in a parent compound is substituted. In addition, the salt may be a pharmaceutically acceptable salt.

As used herein, the term “pharmaceutically acceptable” means approved or approvable by the government or equivalent regulatory agency, or listed in the pharmacopoeia or other generally recognized pharmacopoeia for use in animals, and more particularly in humans, without causing significant toxic effects when used in commonly used medicinal dosages.

As used herein, the term “hydrate” means to a compound to which water is bonded, and refers to a broad concept including an encapsulated compound that does not have a chemical bonding force between water and the compound.

As used herein, the term “solvate” means a higher-order compound formed between a molecule or ion of a solute and a molecule or ion of a solvent.

As used herein, the term “active ingredient” means a component capable of exhibiting the desired activity alone or in combination with a carrier that does not have the activity itself.

As used herein, the term “skin” means a tissue covering the body surface of an animal, and refers to the broadest concept including not only tissues covering body surfaces such as the face or body, but also scalp and hair.

As used herein, the term “skin defense” is the ability to protect the skin from external stimuli and toxins, and the stimulation refers to a broad concept including all harmful stimuli to the skin, such as particulate matter, bacteria, viruses, ultraviolet rays, dryness, and wounds.

In one embodiment, the 2-isopropylmalic acid or the salt, hydrate or solvate thereof by administration of the composition may be administered at a dosage of 1.7 to 27 mg/kg/day. When the dosage of the 2-isopropylmalic acid or the salt, hydrate or solvate thereof by the administration of the composition is less than 1.7 mg/kg/day, it may be difficult to exhibit the effect of enhancing skin elasticity or improving skin wrinkles, and if the dosage is greater than 27 mg/kg/day, it may cause skin irritation. Specifically, the dosage of the 2-isopropylmalic acid or the salt, hydrate or solvate thereof by administration of the composition may be 1.7 mg/kg/day or more. 1.7 mg/kg/day or more, 2 mg/kg/day or more, 2.5 mg/kg/day or more, 3 mg/kg/day or more, 3.5 mg/kg/day or more, 4 mg/kg/day or more, 4.5 mg/kg/day or more, 5 mg/kg/day or more, 5.5 mg/kg/day or more, 6 mg/kg/day or more, 6.5 mg/kg/day or more, 7 mg/kg/day or more, 7.5 mg/kg/day or more, 8 mg/kg/day or more, 8.5 mg/kg/day or more, 9 mg/kg/day or more, 9.5 mg/kg/day or more, 10 mg/kg/day or more, 10.5 mg/kg/day or more, 11 mg/kg/day or more, 11.5 mg/kg/day or more, 12 mg/kg/day or more, 12.5 mg/kg/day or more or 13 mg/kg/day or more, 27 mg/kg/day or less, 26.5 mg/kg/day or less, 26 mg/kg/day or less, 25.5 mg/kg/day or less, 25 mg/kg/day or less, 24.5 mg/kg/day or less, 24 mg/kg/day or less, 23.5 mg/kg/day or less, 23 mg/kg/day or less, 22.5 mg/kg/day or less, 22 mg/kg/day or less, 21.5 mg/kg/day or less, 21 mg/kg/day or less, 20.5 mg/kg/day or less, or 20 mg/kg/day or less.

In one embodiment, the active ingredient may activate primary cilia. In the present specification, activating primary cilia may mean promoting production of primary cilia in a cell or increasing the length of primary cilia.

As used herein, the term “primary cilia” refers to an organelle protruding from a cell body that senses various external stimuli, including chemical stimuli, physical stimuli, light, osmotic pressure, fluid flow, and gravity signals. When the activity of the skin primary cilia is inhibited, skin pigmentation, inflammatory dermatitis, etc. may be induced.

In one embodiment, the active ingredient may increase the production of primary cilia inhibited by particulate matter.

In one embodiment, the active ingredient may inhibit the expression of a matrix metalloproteinase-1 (MMP-1) enzyme.

In one embodiment, the skin wrinkles may be skin wrinkles caused by particulate matter.

As used herein, the term “particulate matter” refers to particulate material that is invisible to our eyes and floats or scatters in the atmosphere for a long time. Specifically, the particle size of the particulate matter may be 10 μm or less (PM 10), more specifically, 2.5 μm or less (PM 2.5). In particular, the particulate material having a particle diameter of 2.5 μm or less (PM 2.5) is referred to as “fine particulate matter”, and the term “particulate matter” is intended to include “fine particulate matter” in this specification.

In one embodiment, the composition may be a cosmetic composition. The cosmetic composition may have a formulation such as softening lotion, astringent lotion, nourishing lotion, nourishing cream, massage cream, eye cream, eye essence, essence, cleansing cream, cleansing lotion, cleansing foam, cleansing water, pack, powder, body lotion, body cream, body essence, body cleaner, hair dye, shampoo, conditioner, hair fixative, hair growth promoter, ointment, gel, cream, patch, spray, powder, and skin adhesion type, but is not limited thereto.

In addition, in each formulation, other components other than the above essential component can be suitably selected and formulated by those skilled in the art without difficulty depending on the type or purpose of use of other external preparations.

The cosmetic composition may be provided in any formulation suitable for topical application. For example, it may be provided in the form of a solution, an emulsion obtained by dispersing an oil phase in an aqueous phase, an emulsion obtained by dispersing an aqueous phase in an oil phase, a suspension, a solid, a gel, a powder, a paste, a microneedle, a foam, or an aerosol composition. Compositions of such formulations can be prepared according to a commonly used method in the art.

The cosmetic composition according to the present specification may further include functional additives and components included in general cosmetic compositions in addition to the compound of the present specification. The functional additive may include a component selected from the group consisting of water-soluble vitamins, oil-soluble vitamins, high-molecular peptides, high-molecular polysaccharides, sphingolipids, and seaweed extract. The cosmetic composition according to the present specification may include other ingredients that can give a synergistic effect to the main effect, preferably within a range that does not impair the main effect. In addition, the cosmetic composition according to the present specification may further include a moisturizing agent, an emollient, a surfactant, a UV absorber, a preservative, a bactericide, an antioxidant, a pH adjuster, an organic and inorganic pigment, a fragrance, a cooling agent or an antiperspirant. The blending amount of the components can be easily selected by those skilled in the art within the range that does not impair the purpose and effect of the present specification, and the blending amount may be 0.001 to 10% by weight, specifically 0.01 to 3% by weight, based on the total weight of the composition.

In one embodiment, the composition may be a food composition. The formulation of the food composition is not particularly limited, and for example, it may be formulated as tablet, granule, pill, powder, liquid such as drink, caramel, gel, bar, tea bag, and the like. In the food composition of each formulation, ingredients commonly used in the field other than the active ingredient can be appropriately selected depending on the formulation or purpose of use and formulated by those skilled in the art without difficulty, and when applied simultaneously with other ingredients, a synergistic effect can occur.

The food composition according to one embodiment includes various nutrients, vitamins, minerals (electrolytes), flavoring agents such as synthetic and natural flavoring agents, coloring agents and extenders (cheese, chocolate, etc.), pectic acid and salts thereof, alginic acid and salts thereof, organic acids, protective colloidal thickeners, pH adjusters, stabilizers, preservatives, glycerin, alcohols, carbonation agents used in carbonated beverages, and the like. In addition, the food compositions according to an embodiment may include natural fruit juice and pulp for the production of fruit juice beverages and vegetable beverages. These components may be used independently or in combination. The proportion of these additives is not so important, but is generally included in the range of 0 to about 50 parts by weight per 100 parts by weight of the composition according to an embodiment.

In one embodiment, the composition may be a pharmaceutical composition. The pharmaceutical composition may be a composition for preventing or treating atopic dermatitis, psoriasis, eczema or flushing. The pharmaceutical composition may be administered orally, parenterally, rectally, topically, transdermally, intravenously, intramuscularly, intraperitoneally, subcutaneously, and the like. Formulations for oral administration may be tablets, pills, soft and hard capsules, granules, powders, fine granules, liquids, emulsions, or pellets, but are limited thereto. The formulations for parenteral administration may be solutions, suspensions, emulsions, gels, injections, drops, suppositories, patches, or sprays, but are not limited thereto. The formulation can be easily prepared according to a commonly used method in the art, and surfactants, excipients, wetting agents, emulsification accelerators, suspending agents, salts or buffers for osmotic pressure control, coloring agents, spices, stabilizers, preservatives, preservatives or other commercially available adjuvants may be further included.

In another aspect, the present disclosure provides a composition for activating skin primary cilia including 2-isopropylmalic acid or a salt, hydrate or solvate thereof as an active ingredient.

In one embodiment, the activation of the skin primary cilia may be the activation of the skin primary cilia inhibited by particulate matter. When the skin is exposed to particulate matter, the production of skin primary cilia is suppressed and the length is reduced. The composition according to the present disclosure prevents the production of skin primary cilia from being inhibited by particulate matter as described above, thereby enhancing the production of skin primary cilia, and increasing the length of skin primary cilia exposed to particulate matter.

In one embodiment, the active ingredient may enhance the production of skin primary cilia.

In another aspect, the present disclosure provides a composition for enhancing skin defense against particulate matter including 2-isopropylmalic acid or a salt, hydrate or solvate thereof as an active ingredient.

In another aspect, the present disclosure provides the use of 2-isopropylmalic acid or a salt, hydrate or solvate thereof for preparing a composition for enhancing skin elasticity or improving skin wrinkles.

In another aspect, the present disclosure provides a method for enhancing skin elasticity or improving skin wrinkles including a step of administering to a subject in need of enhancing skin elasticity or improving skin wrinkle a composition including an effective amount of 2-isopropylmalic acid or a salt, hydrate or solvate thereof.

In another aspect, the present disclosure provides the use of 2-isopropylmalic acid or a salt, hydrate or solvate thereof for the preparation of a composition for activating skin primary cilia.

In another aspect, the present disclosure provides a method for activating skin primary cilia including the step of administrating to a subject in need of activating skin primary cilia a composition including an effective amount of 2-isopropylmalic acid or a salt, hydrate or solvate thereof.

In another aspect, the present disclosure provides the use of 2-isopropylmalic acid or a salt, hydrate or solvate thereof for preparing a composition for enhancing skin defense against particulate matter.

In another aspect, the present disclosure provides a method for enhancing skin defense against particulate matter including the step of administering to a subject in need of enhancing skin defense against particulate matter a composition including an effective amount of 2-isopropylmalic acid or a salt, hydrate or a solvate thereof.

Hereinafter, the present invention will be described in more detail through examples and the like. These examples are only for illustrating the present invention, and it will be apparent to those of ordinary skill in the art that the scope of the present invention is not to be construed as being limited by these examples.

### [Experimental Example 1] Cytotoxicity Measurement

Human-derived fibroblasts were placed in DMEM (Dulbecco's Modified Eagle's medium) containing 10% fetal bovine serum (FBS) and cultured at 37° C. under 5% CO₂ conditions. The cultured human-derived fibroblasts were treated with 2-isopropylmalic acid at different concentrations (1 μM, 10 μM, 100 μM, 1000 μM) for 24 hours. CCK-8 reagent (Donjindo Inc., CK04) was diluted 1/10 in DMEM medium, treated for 3 hours, and absorbance was measured at 450 nm, and the results were shown in FIG. 1.

### [Experimental Example 2] Assessment of Primary Ciliary Activation

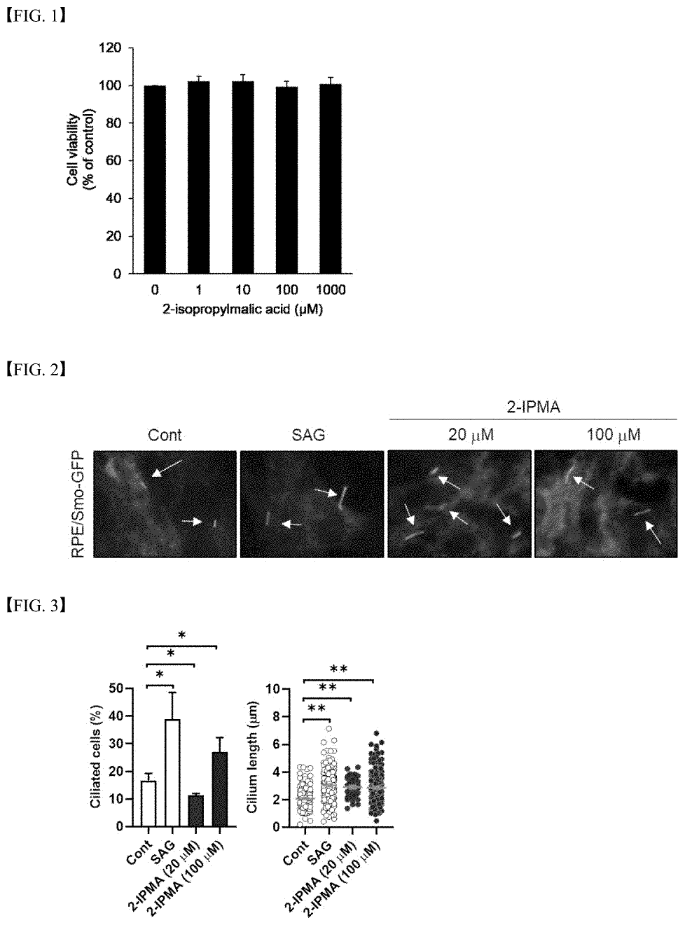

In order to confirm the primary cilia activation effect of 2-isopropylmalic acid, RPE/Smo-GFP, primary cilia model cells, was prepared. Specifically, human telomerase-immortalized retinal pigmented epithelial (RPE) cells (ATCC Inc.) were transformed with Smo-GFP plasmid (Addgene Inc.) to prepare RPE/Smo-GFP. After treating RPE/Smo-GFP with 2-isopropylmalic acid (Sigma Aldrich Inc.) at different concentrations (20 μM, 100 μM) for 24 hours, the primary cilia production rate and primary cilia length were measured by confocal microscopy (LSM510; Carl Zeiss Microimaging Inc., Thornwood, N.Y.) and the results were shown in FIGS. 2 and 3. The production rate of primary cilia was expressed as the ratio of the number of cells having primary cilia to the total number of cells as a percentage, and the length of primary cilia was a measure of the actual length of all cilia observed under a microscope. As a positive control, SAG (SMO agonist, Calbiochem Inc.), which was a primary cilia production promoting substance, was used.

The white arrow in FIG. 2 indicates primary cilia. As can be seen in FIGS. 2 and 3, it was confirmed that when 2-isopropylmalic acid (2-IMPA) was treated, the proportion of cells with primary cilia was statistically significantly higher than that of the control group, and the length of the cilia was also increased.

### [Experimental Example 3] Assessment of Activation of Primary Cilia Inhibited by Particulate Matter

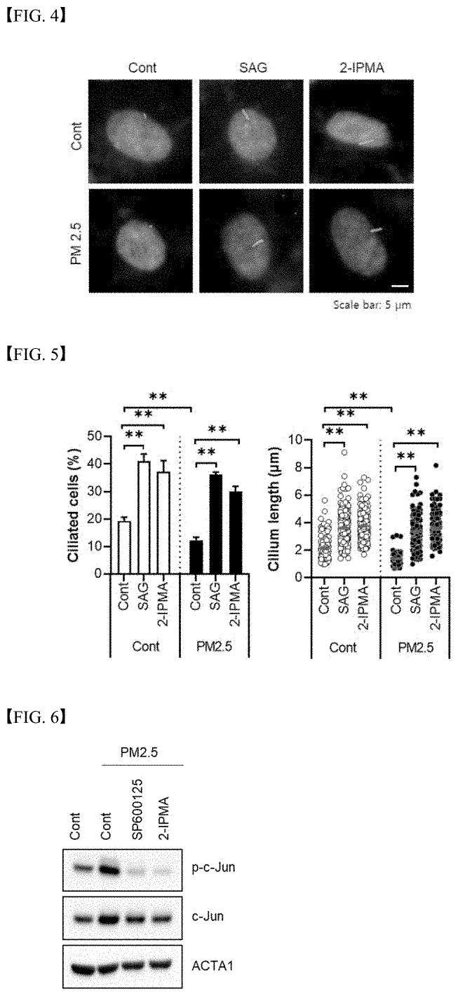

Particulate matter (PM2.5) was treated in RPE/Smo-GFP, which was the primary cilia model cells of Experimental Example 2. Specifically, particulate matter was directly collected in the air (37.34° N, 127.27° E, 167 m above sea level) using a Teflon filter (Teflon filter, Zefluor, Pall Life Science, Ann Arbor, Mich., USA). Then, this was treated at a concentration of 50 ug/ml in the cell culture medium. Thereafter, after treatment with 100 μM of 2-isopropylmalic acid for 24 hours, the fluorescence of Smo-GFP was visually observed using a fluorescence microscope, and then the production rate and length of primary cilia were measured and shown in FIGS. 4 and 5. The production rate of primary cilia was expressed as the ratio of the number of cells having primary cilia to the total number of cells as a percentage, and the length of primary cilia was a measure of the actual length of all cilia observed under a microscope. As a positive control, SAG (SMO agonist, Calbiochem Inc.), which was a primary cilia production promoting substance, was used.

As can be seen in FIGS. 4 and 5, it was confirmed that when particulate matter was treated, the production of primary cilia was suppressed, but when 2-isopropylmalic acid was treated, the production of primary cilia suppressed by particulate matter was restored, and the length of primary cilia was also getting longer.

### [Experimental Example 4] Assessment of Changes in Expression of Biomarkers

Human-derived fibroblasts (LONZA Inc.) were treated with particulate matter (PM2.5). Then, after treatment with 100 μM of 2-isopropylmalic acid for 24 hours, cell lysate was made, protein was quantified, and the same amount of protein was separated by electrophoresis, and c-Jun, p-c-Jun (phosphorylated c-Jun) was performed by western blotting. MMP-1 and collagen-specific antibodies were used to measure the expression of each protein in an antigen-antibody reaction, and the results were shown in FIGS. 6 to 8, respectively.

As can be seen in FIG. 6, it was confirmed that when particulate matter was treated, phosphorylation of c-Jun was promoted and the amount of p-c-Jun was increased, but when 2-isopropylmalic acid was treated, phosphorylation of c-Jun by particulate matter was suppressed. This effect was similar to SP600125 (Sigma Aldrich Inc.), a substance known to inhibit phosphorylation of c-Jun. When phosphorylation of c-Jun was promoted, primary cilia production was inhibited, and thus, it could be confirmed that 2-isopropylmalic acid inhibited phosphorylation of c-Jun, thereby activating the primary cilia.

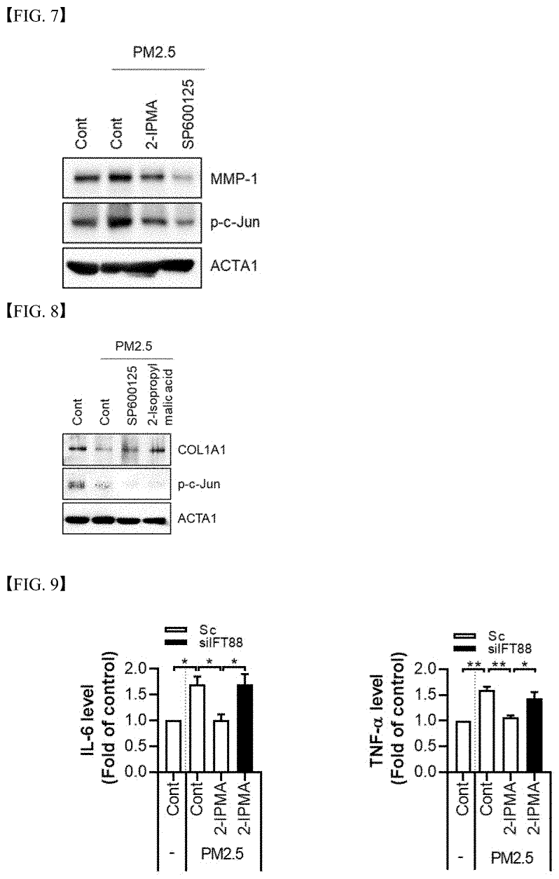

As can be seen in FIGS. 7 and 8, when particulate matter was treated, the expression of MMP-1, an enzyme that decomposes collagen, was increased and collagen was decreased. However, it was confirmed that when 2-isopropylmalic acid was treated, MMP-1 was decreased and collagen was increased.

### [Experimental Example 5] Assessment of Expression Changes of IL-6 and TNF-α

Human-derived fibroblasts (LONZA Inc.) were transfected with siRNA for IFT88 (siIFT88) or scrambled siRNA (SC), respectively, and then were treated with 50 ug/mL of particulate matter (PM2.5) and 100 μM of 2-isopropylmalic acid for 24 hours. Thereafter, the expression levels of IL-6 and TNF-α, which were inflammatory cytokines, were measured in culture conditions by ELISA assay, and the results were shown in FIG. 9.

As can be seen in FIG. 9, it was confirmed that when particulate matter was treated, the expression of IL-6 and TNF-α was increased, but when 2-isopropylmalic acid was treated, the expression of IL-6 and TNF-α increased by particulate matter was reduced. In addition, in the group transfected with siRNA (siIFT88) that depleted IFT88, which played an important role in primary cilia production, the expression of IL-6 and TNF-α was high. Through these results, it was confirmed that 2-isopropylmalic acid had an anti-inflammatory effect by promoting the production of primary cilia in fibroblasts treated with particulate matter.

### [Experimental Example 6] Assessment of Inhibition of MMP-1 Expression in an Artificial Skin Model

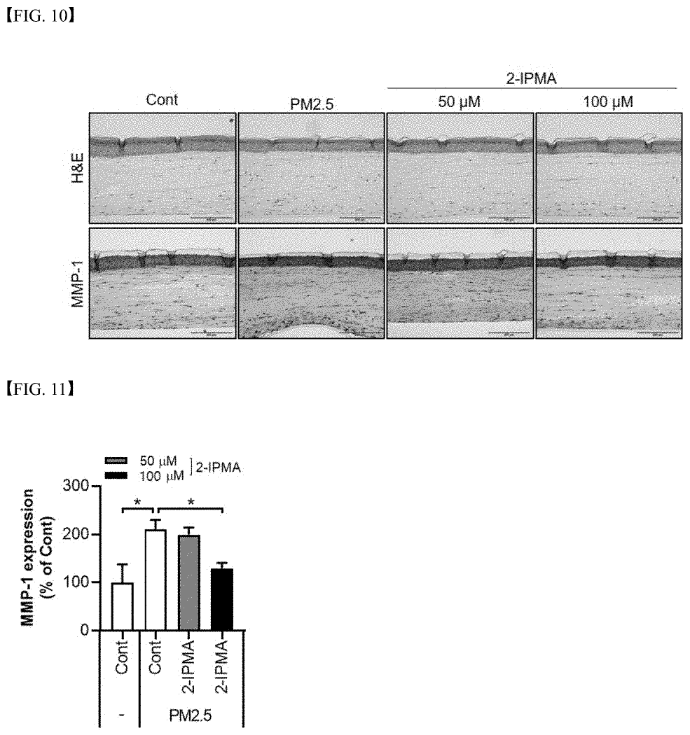

The effect of 2-IPMA on the skin was evaluated using a full-thickness human three-dimensional (3-D)-skin model. The full-thickness human 3D-skin model was fabricated in the following way. An artificial dermal layer was formed by loading a mixture of collagen (Nutragen, Advanced Biomatrix Inc.) and fibroblasts (NDFn, ThermoFisher Inc.) in a volume of 400 ul in Transwell® (Corning Inc.). Thereafter, a fibroblast culture medium (M106, Gibco Inc.) was put on the artificial dermal layer and cultured for 7 days to stabilize the artificial dermal layer. Next, keratinocytes (HEKn, ThermoFisher Inc.) were seeded on the artificial dermal layer, and cultured in keratinocyte culture medium (EpiLife, Gibco Inc.) for 1 day, followed by epidermal layer production medium (CnT-3DPR, CellnTEC) for 1 day, and then cultured in the same epidermal layer production medium for 10 days in a state of exposure to air to form an artificial epidermal layer, thereby constructing a full-thickness human 3D-skin model. 100 ug/mL of particulate matter (PM2.5) and 2-isopropylmalic acid (50 uM, 100 uM) were treated in the full-thickness human three-dimensional skin model constructed in this way for 48 hours. Thereafter, hematoxylin-eosin (H&E) staining and MMP-1 immunocytochemistry were performed, and the results were shown in FIG. 10. In addition, the expression level of MMP-1 was quantified through image analysis and shown in FIG. 11.

As can be seen in FIGS. 10 and 11, it was confirmed that the 3D skin model had a normal skin structure composed of fibroblasts including the well-stratified epidermis and dermis through the H&E staining results, and when treated with only particulate matter, the expression of MMP-1 was increased, but when treated with 2-isopropylmalic acid, the expression of MMP-1 increased by the particulate matter was decreased.

### [Formulation Example 1] Liquid Ampoule

A liquid ampoule was prepared with the composition shown in Table 1 below according to a commonly used method.

TABLE 1

| | Contents |
|---|---|
| Ingredients | (wt %) |
| 2-isopropylmalic acid | 3.2 |
| Arginine | 0.50 |
| Green tea polyphenol | 0.05 |
| Indigestible maltodextrin | 1.00 |
| Vitamin C | 0.10 |
| Citric acid | 0.10 |
| Fragrance | 0.05 |
| Purified water | Balance |
| Total | 100.00 |

### [Formulation Example 2] Soap

A soap was prepared with the composition shown in Table 2 below according to a commonly used method.

TABLE 2

| | Contents |
|---|---|
| Ingredients | (wt %) |
| 2-isopropylmalic acid | 3.00 |
| Titanium dioxide | 0.20 |
| Polyethylene glycol | 0.80 |
| Glycerin | 0.50 |
| Ethylenediaminetetraacetic acid | 0.05 |
| Sodium | 1.00 |
| Pigment | 0.20 |
| Soapy scent | 0.20 |
| Soap base | Balance |
| Total | 100.00 |

### [Formulation Example 3] Lotion

A lotion was prepared with the composition shown in Table 3 below according to a commonly used method.

TABLE 3

| | Contents |
|---|---|
| Ingredients | (wt %) |
| 2-isopropylmalic acid | 3.00 |
| L-ascorbic acid-2-magnesium phosphate salt | 1.00 |
| Collagen | 1.00 |
| Sodium citrate | 0.10 |
| Citric acid | 0.05 |
| Licorice Extract | 0.20 |
| 1,3-butylene glycol | 3.00 |
| Purified water | Balance |
| Total | 100.00 |

### [Formulation Example 4] Cream

A cream was prepared with the composition shown in Table 4 below according to a commonly used method.

TABLE 4

| | Contents |
|---|---|
| Ingredients | (wt %) |
| 2-isopropylmalic acid | 3.00 |
| Polyethylene glycol monostearate | 2.00 |
| Self-emulsifying glycerin monostearate | 5.00 |
| Cetyl alcohol | 4.00 |
| Squalene | 6.00 |
| Tri2-glyceryl ethyl henate | 6.00 |
| Sphingoglycolipids | 1.00 |
| 1,3-butylene glycol | 7.00 |
| Purified water | Balance |
| Total | 100.00 |

### [Formulation Example 5] Pack

A pack was prepared with the composition shown in Table 5 below according to a commonly used method.

TABLE 5

| | Contents |
|---|---|
| Ingredients | (wt %) |
| 2-isopropylmalic acid | 3.00 |
| Polyvinyl alcohol | 13.00 |
| L-ascorbic acid-2-magnesium phosphate salt | 1.00 |
| Lauroylhydroxyproline | 1.00 |
| Collagen | 2.00 |
| 1,3-butylene glycol | 3.00 |
| Ethanol | 5.00 |
| Purified water | Balance |
| Total | 100.00 |

### [Formulation Example 6] Cosmetic Liquid Formulation

A cosmetic liquid formulation was prepared with the composition shown in Table 6 below according to a commonly used method.

TABLE 6

| | Contents |
|---|---|
| Ingredients | (wt %) |
| 2-isopropylmalic acid | 3.00 |
| Hydroxyethylene Cellulose | 12.00 |
| xanthan gum | 2.00 |
| 1,3-butylene glycol | 6.00 |
| Glycerin | 4.00 |
| Sodium Hyaluronate | 5.00 |
| Purified water | Balance |
| Total | 100.00 |

### [Formulation Example 7] Soft Capsules

100 mg of 2-isopropylmalic acid, 160 mg of L-carnitine, 320 mg of soybean oil, 2 mg of palm oil, 8 mg of hydrogenated vegetable oil, 4 mg of yellow wax and 6 mg of lecithin were mixed and filled into one capsule according to a commonly used method to prepare a soft capsule.

### [Formulation Example 8] Tablet

100 mg of 2-isopropylmalic acid, 500 mg of galactooligosaccharide, 80 mg of lactose, and 220 mg of maltose were mixed and granulated using a fluid bed dryer. Then, 6 mg of sugar ester was added to the mixture and was compressed with a tablet press to prepare tablets.

### [Formulation Example 9] Granules

100 mg of 2-isopropylmalic acid, 250 mg of anhydrous crystalline glucose and 550 mg of starch were mixed, formed into granules using a fluid bed granulator, and then filled in a bag to prepare granules.

### [Formulation Example 10] Drink Formulation

80 mg of 2-isopropylmalic acid, 10 g of glucose, 0.6 g of citric acid, and 25 g of liquid oligosaccharide were mixed. Then, 500 ml of purified water was added to the mixture, and the mixture was filled in each bottle by 200 ml. After filling the bottle, it was sterilized at 130° C. for 4 to 5 seconds to prepare a drink beverage.

1. A method for enhancing skin elasticity or improving skin wrinkles, comprising the step of administering to a subject in need of enhancing skin elasticity or improving skin wrinkles a composition comprising an effective amount of 2-isopropylmalic acid or a salt, hydrate or solvate thereof.

2. The method according to claim 1, wherein a dosage of the 2-isopropylmalic acid or the salt, hydrate or solvate thereof is 1.7 to 27 mg/kg/day.

3. The method according to claim 1, wherein the composition activates primary cilia.

4. The method according to claim 1, wherein the composition inhibits an expression of an MMP-1 enzyme.

5. The method according to claim 1, wherein the skin wrinkles are skin wrinkles caused by particulate matter.

6. The method according to claim 1, wherein the composition is a food composition.

7. The method according to claim 1, wherein the composition is a cosmetic composition.

8. A method for activating skin primary cilia comprising the step of administering to a subject in need of activating skin primary cilia a composition comprising an effective amount of 2-isopropylmalic acid or a salt, hydrate or solvate thereof.

9. The method according to claim 8, wherein the activation of the skin primary cilia is the activation of the skin primary cilia inhibited by particulate matter.

10. The method according to claim 8, wherein the composition enhances a production of the skin primary cilia,

11. The method according to claim 8, wherein a dosage of the 2-isopropylmalic acid or the salt, hydrate or solvate thereof is 1.7 to 27 mg/kg/day.

12. The method according to claim 8, wherein the composition is a food composition.

13. The method according to claim 8, wherein the composition is a pharmaceutical composition.

14. The method according to claim 8, wherein the composition is a cosmetic composition.

15. A method for enhancing skin defense against particulate matter, comprising the step of administering to a subject in need of enhancing skin defense against particulate matter a composition comprising an effective amount of 2-isopropylmalic acid or a salt, hydrate or solvate thereof.

16. The method according to claim 15, wherein a dosage of the 2-isopropylmalic acid or the salt, hydrate or solvate thereof is 1.7 to 27 mg/kg/day.
